(19) United States
(12) Patent Application Publication
(10) Pub. No.: US 2016/0022639 A1
(43) Pub. Date: Jan. 28, 2016

(54) FIXED DOSE COMBINATION FOR PAIN RELIEF WITHOUT EDEMA

(71) Applicant: Autotelic LLC, City of Industry, CA (US)

(72) Inventor: Vuong Trieu, Agoura Hills, CA (US)

(21) Appl. No.: 14/798,737

(22) Filed: Jul. 14, 2015

Related U.S. Application Data
(63) Continuation-in-part of application No. PCT/US2015/011148, filed on Jan. 13, 2015.
Application No. PCT/US2015/011148 is a continuation-in-part of application No. PCT/US2015/034706, filed on Jun. 8, 2015.
Application No. PCT/US2015/034706 is a continuation-in-part of application No. PCT/US2015/034738, filed on Jun. 8, 2015.
(60) Provisional application No. 62/023,962, filed on Jul. 14, 2014.

(51) Int. Cl. A61K 31/415; A61K 31/40; A61K 31/4155

(52) U.S. Cl. CPC A61K 31/415; A61K 31/4155; A61K 31/40

(57) ABSTRACT

Compositions and methods for individualized therapy of pain using a non-steroidal anti-inflammatory drug (NSAID) and an anti-hypertensive without inducing intolerable edema. Said methods comprise basing NSAID/anti-hypertensive dose on each patient's pharmacokinetic response to said NSAID.

### CROSS-REFERENCE TO RELATED APPLICATIONS

This patent application claims the benefit of U.S. Provisional Patent Application No. 62/023,962, filed Jul. 14, 2014, and also claims the benefit of PCT Application No. PCT/US2015/011148, filed Jan. 13, 2015, PCT application No. PCT/US2015/034738, filed Jun. 8, 2015, and PCT/US2015/034706, filed Jun. 8, 2015, all three of which are incorporated by reference, the entire disclosures of which are hereby incorporated by reference.

### STATEMENT REGARDING FEDERALLY SPONSORED RESEARCH AND DEVELOPMENT

This invention was made without Government support

### INCORPORATION-BY-REFERENCE OF MATERIAL SUBMITTED ELECTRONICALLY

Does not apply.

### BACKGROUND OF THE INVENTION

It is well appreciated that non-steroidal anti-inflammatory drugs (“NSAID”) are highly active as pain relievers. However, NSAIDs also have major and minor side effects. One of these side effects is drug induced edema.

“Edema” is an abnormal accumulation of fluid in the tissue spaces, cavities, or joint capsules of the body, which causes swelling of the area. Edema can occur in the tissues or body spaces such as the plural cavities or the peritoneal space. Clinically, edema has a variety of consequences depending on the site and severity of the edema. In contrast, chronic, severe subdermal edema can cause skin break down, ulceration and serious infection. Similarly, while a pleural effusion may spontaneously resolve, ascites (edema in the paritoneal space) can be complicated by difficulty to treat bacterial peritonitis. See, e.g., Harrison's Internal Medicine, 16^{th} edition, p. 213-214.

Pathophysiologically, edema occurs when there is an elevation in capillary hydraulic pressure, and/or an increase in capillary permeability or when the interstitial oncotic pressure exceeds the plasma oncotic pressure. Such changes can result from a variety of conditions and diseases. For example, in congestive heart failure, the activation of the renin-angiotensin system causes volume overload which results in increased capillary hydraulic pressure. The kidneys control extracellular fluid volume by adjusting sodium and water excretion. When renal function is impaired, edema can result. In cirrhosis the reduced production of serum proteins such as albumin result in a decrease in the plasma oncotic pressure relative to interstitial oncotic pressure resulting fluid shifts into the intersitum. Venous insufficiency is a common cause of edema of the lower extremities from an increase in capillary hydraulic pressure. Harrison's Internal Medicine, 16^{th} edition, p.213-214; O'Brian et al. Treatment of Edema, American Family Physician, 71(11). 2111-17.

Many drugs can cause edema including steroid hormones, vasodilators such as hydralazine, estrogens, NSAIDs, immunomodulators such as interleukin 2, and calcium channel blockers. The pathophysiology of drug induced edema is similarly wide ranging. Such mechanisms include, drug induced vasodilation (e.g., hydralazine), drug effects on the kidneys' sodium excretion (e.g., steroids), and capillary damage (e.g., interleukin 2). Drug induced edema is usually dose-dependent and its severity increases over time. Harrison's Internal Medicine, 16^{th} edition, p.213-214; O'Brian et al., Treatment of Edema, American Family Physician, 71(11). 2111-17.

Many NSAIDs can cause edema. The mechanism for NSAID induced edema has been postulated to be from renal vasoconstriction. Harrison's Internal Medicine, 16th edition, p.213-214. NSAIDs inhibit cyclogenases (COX), the enzymes that catalyze formation of various prostaglandins. The two principle COX isoforms are COX-1 and COX-2. Studies have shown that both therapeutic and side effects of NSAIDs are dependent on cyclooxygenase inhibition. In general, selective inhibitors of COX-2 have therapeutic effects that are as strong as conventional NSAIDs but with fewer side effects. Nevertheless, selective COX-2 inhibitors still can cause edema. Suleyman et al., Anti-inflammatory and side effects of cyclooxygenase inhibitors, Pharma. Reports, 2007 59:247-258.

Celebrex (celecoxib) is a prototypic selective COX-2 inhibitor and the first page of the Celebrex (celecoxib) Package Insert lists edema as an “adverse reaction.” Table 1 of this Package Insert discloses that 2.1% of patients treated with celecoxib develop edema, as compared to 1.1%, 2.1%, 1.0%, and 3.5% for placebo, naproxen, diclofenac, and ibuprofen, respectively. Moore et al.'s review of the tolerability and rate of adverse events in clinical trials of celecoxib found that the incidence of edema at any site was usually about 3%, but in two trials the incidence of edema much higher at 23% and 38%. Arthritis Res. & Therapy, 2005, 7(6), R644-R664, R658-59.

In view of the persistent problem of drug induced edema and, in particular, edema induced by drugs with known efficacy for the treatment of pain, there remains a need for better approaches for preventing and treating drug induced edema.

### BRIEF SUMMARY OF THE INVENTION

The invention provides a composition for treating pain without inducing edema comprising a NSAID and an anti-hypertensive, wherein the composition is administered in a fixed-dose combination.

Additionally, provided is a method for individualized therapy of pain using a NSAID, wherein the NSAID can be a COX-2 inhibitor, said method comprising wherein an individual takes direct action to cause or otherwise causes:
a. administering, or causing the administration of, a first NSAID formulation comprising a first dose of a NSAID in combination with an anti-hypertensive in specific amounts, to a first patient suffering from pain;
b. determining, or causing the determination of the NSAID and anti-hypertensive concentrations in the first patient's blood at a plurality of time data points after the first NSAID formulation has been administered to the first patient;
c. transforming or, causing the transformation of the first patient's NSAID and anti-hypertensive concentration/time data points into one or more pharmacokinetic (PK) parameters;
d. a comparison the first patient's values for said PK parameters to predetermined ranges of values for each PK parameter and if one or more of the first patient's PK parameters fall outside of a predetermined range, designing a new NSAID formulation, wherein the dose of said NSAID inhibitor, the anti-hypertensive, or both is different from that of the first NSAID formulation;
e. administration of the new NSAID formulation to the first patient;
f. repeating steps b-e until all the PK parameters used in step d are within said predetermined ranges, and
g. if pain control is adequate, edema is tolerable, and the patient is not experiencing edema, maintaining the first patient on the NSAID formulation Further, the invention provides methods for predicting the response to therapy of pain using a NSAID, wherein the NSAID may be a COX-2 inhibitor said method comprising:
a. administering, or causing the administration of a first NSAID formulation comprising a first dose of a NSAID in combination with an anti-hypertensive in specific amounts, to a first patient suffering from pain;
b. determining, or causing the determination of, the NSAID and anti-hypertensive concentration in the first patient's blood at a plurality of time data points after the first NSAID formulation has been administered to the first patient;
c. transforming, or causing the transformation of, the first patient's NSAID and anti-hypertensive concentration/time data points in to one or more pharmacokinetic (PK) parameters;
d. comparing the first patient's values for said PK parameters to a predetermined ranges of values for each PK parameter and if one or more of the first patient's PK parameters fall outside of a predetermined range, with deviation from the desired range being predictive of inefficacy or increased toxicity. The invention also provides methods of data mining, particularly data reduction and clustering protocols related to the said “predetermined ranges” of PK parameters. In preferred embodiments of the compositions and methods of the invention, the NSAID is celecoxib and the anti-hypertensive agent is either a Angioensin receptor blocker or an ACE inhibitor

Related compositions and methods for individualizing therapy of arthritic pain are disclosed herein.

### BRIEF DESCRIPTION OF THE DRAWINGS

FIG. 1 is a scatterplot graph displaying the relationship between LPS-stimulated plasma PGE2 ex vivo, an index of NSAID activity, and log plasma concentrations of celecoxib 2, 4, 6, and 24 hours after dosing. PGE2 is expressed as a percentage of predosing values. A steep but variable dose-response is evident. (P, 0.01 vs. placebo) (from McAdam et al. Systemic biosynthesis of prostacyclin by cyclooxygenase (COX)-2: the human pharmacology of a selective inhibitor of COX-2. PNAS. 1999; 96:272-7.)

FIG. 2 depicts the pharmacokinetic parameters produced by different doses of celecoxib that were administered to subjects.

FIG. 3 displays the result of a meta-analysis of the one dose AUC from patients in different age groups.

FIG. 4 displays the result of a meta-analysis of the celecoxib dose dependence of edema.

FIG. 5 displays the result of a regression analysis of the celecoxib dose dependence of edema.

FIGS. 6-11 display the incidence of edema for celecoxib given alone or with other drugs. HCTZ, hydrochlorothiazide; CCB, calcium channel blocker; ARB, angiotensin receptor blocker; ACE, angiotensin converting enzyme inhibitor; non-thiazide diuretic; beta blocker; any, any other drug.

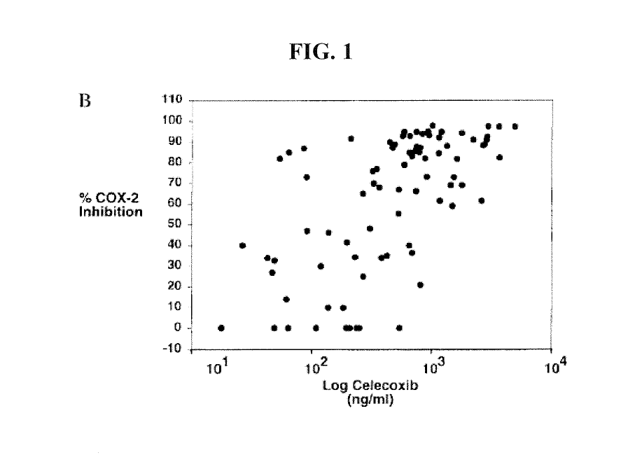

### DETAILED DESCRIPTION OF THE INVENTION

The invention provides compositions and methods for individualized therapy of pain, including but not limited to arthritic pain, using a NSAID, preferably a COX-2 inhibitor, and an anti-hypertensive (e.g., lisinopril or olmesartan). The high patient to patient variability in response to a dose of any NSAID and/or anti-hypertensive makes the mere clinical monitoring of patients an inadequate way to treat patients with these classes of drugs. Even the measurement of “blood levels” (i.e., the occasional measurement of the drug's concentration in the plasma) is unlikely to lead to effective nontoxic regimens. Given the complexity of NSAIDs' dose response relationships both alone and in combination with one or more anti-hypertensives, a more comprehensive set of metrics must employed in each patient in order more efficaciously address a patient's pain. The methods claimed herein take advantage of a pharmacokinetic (“PK”) analysis for each patient. As such, the claimed methods go beyond the conventional methods for drug monitoring which often involve measurement of a single blood level at a single time point. Instead, the claimed methods make use of data of the plasma drug concentration from several time points (e.g., at least 2 time points, and preferably at least 12 over 72 hours) and take advantage of the full scope of PK parameters to generate an individualized PK Profile of a patient that is unique to a particular drug or a combination of drugs. There is no known method of predicting the individual PK profile for celecoxib in an individual patient due to the high variability and complexity of human pharmacokinetics. As such there is no natural law known that can explain human pharmacokinetics. Therefore, the claimed method seeks to determined the individual's PK profile directly.

As used herein the phrase “individualized therapy” refers to a specific treatment regimen for a patient comprising the administration of one or more drugs, which is the result of analyzing the individualized pharmacokinetic and/or pharmacodynamic parameters of the subject to maximize drug efficacy at the lowest dosage of the drug(s) possible.

As used herein “pain” refers to physical suffering or discomfort caused by an illness or injury, e.g., arthritis.

As used herein “osteoarthritic pain” refers to pain resulting from osteoarthritis.

As used herein, “formulation” or “a formulation” refers to a combination of active ingredients and pharmaceutically acceptable carriers wherein each is present in a dosage form at fixed ratios to one another (i.e., fixed percentages of each ingredient in the dosage form.)

As used herein, “first NSAID formulation” or “first NSAID formulation” is the NSAID formulation comprising a first dose of a NSAID combined with a dose of anti-hypertensive and, optionally, pharmaceutically acceptable carriers in specific amounts that is administered to the patient to begin the process by which PK parameters are determined. The “new NSAID inhibitor formulation” is the formulation designed based on the PK parameters produced by the first NSAID inhibitor formulation. As such, the “new NSAID inhibitor formulation” or “second NSAID formulation” may have a change in the dose of the NSAID inhibitor, the dose of the anti-hypertensive or the composition of the carrier. Alternatively, the composition of the carrier may be changed while the doses of the NSAID inhibitor and the anti-hypertensive remain the same. To evaluate the PK parameters produced by the new NSAID inhibitor formulation it replaces the “first NSAID inhibitor formulation” and the process is repeated. The new NSAID inhibitor formulation comprising a first dose of a NSAID combined with an anti-hypertensive in specific amounts formulation may be a second formulation.

As used herein the phrases “first formulation under a first regimen” and “first formulation” refer to the dosage of a NSAID or COX-2 inhibitor formulation that an individual initially receives prior to performing one or more steps of the claimed invention. For example, the first formulation under a first regimen, can be the standard 100 mg or 200 mg dosages of celecoxib prescribed to patients over 60 kg, twice daily for osteoarthritis combined with a standard dose of anti-hypertensive.

As used herein, the phrase “NSAID and anti-hypertensive concentration/time data points” refers to the NSAID and anti-hypertensive concentration in a unit of volume (e.g., 1 ml) of plasma from a subject at a given point in time before or after administration of the NSAID and anti-hypertensive.

As used herein, the phrase “transforming” the patient's NSAID and anti-hypertensive concentration/time data points” refers to the application of mathematical operations, formulas, theories, and/or principles to the NSAID or anti-hypertensive concentrations/time data points of an individual to derive PK parameters (e.g., a formula for calculating AUC).

As used herein, the phrase “predetermined range of values” refers to a range of PK parameters associated with desirable drug efficacy with minimal toxicity determined by a statistical analysis the individualized PK profiles of patients of known outcomes for the formulation administered. The predetermined range of values can readily be compared to a patient's values and used to adjust dosages.

As used herein, “Significant adverse drug reactions” refer to ADRs that the patient finds intolerable, impair physiologic functions, and/or put the patient at risk for immobility and/or death or combinations thereof.

As used herein “Significant side effects” refer to side effects that the patient finds intolerable, impair physiologic functions, and/or put the patient at risk for immobility and/or death or combinations thereof.

As used herein, the phrase “predetermined range of values” refers to a range of PK parameters associated with desirable drug efficacy with minimal toxicity determined by a statistical analysis of the “PK profiles” (collections of PK parameters) of patients of known outcomes for the formulations administered.

A variety of calculations can be used in accordance with the current invention. Exemplary calculations useful in methods of the invention include discriminant analysis, in which a new individual is classified from known calculations by training with a set of individuals of known classification. Data from individuals with known health states can be used to classify a new individual as having one of these known health states. Other exemplary methods include classification analysis, which is similar to discriminant analysis, and multiple discriminant analysis.

The identification of said “predetermined ranges” of values can also be accomplished with the aid of data reduction (e.g., factor analysis or principal components analysis) and clustering computer protocols (e.g., K-means or SOMs). Cluster analysis can be used to find groups, for example, to group disease-associated PK profiles or to cluster individuals into groups of different health states. As such, cluster analysis can be used to identify PK parameters from among all of the PK parameters determined, that are associated with a disease state or condition (e.g., pain, edema and the like) or indicative of a particular disease or progression of a disease or the nature of response to a drug or combination of drugs.

For example, U.S. Pat. No. 7,343,247 (the '247 patent) is directed to methods of classifying drug responsiveness using multiparameter analysis. Unlike the current invention which analyzes pharmacokinetic parameters, the '247 patent teaches comparing the expression levels of specific of molecules in a specimen from the first individual with a health-associated reference expression profile. From a data analysis point of view, the molecule levels used in the '247 patent can be replaced with the PK parameters that are determined in the current application.

In another exemplary approach to data analysis which can be PK profiles, U.S. Pat. No. 8,999,648 discloses a system for classifying a biological sample from a cancer patient and calls for at least one statistical classification program such as of k-nearest neighbors (kNN), linear discriminant analysis, principal components analysis, nearest centroid classification, and support vector machines. (See also, U.S. Pat. Nos. 7,412,333; 8,660,370; 8,990,135; 9,002,658; 9,026,536; and 9,043,321—all of which can be applied to PK profiles)

Other distinct analytic techniques that can be used on PK profiles in accordance with the invention to identify “predetermined ranges.” Analysis of variance (ANOVA) is a general statistical technique useful for testing the significance of differences between and among groups. Regression tree analysis is a predictive method based on a tree structure trained from a set of data. Training is carried out with a series of decisions. For example, a first decision can be if a PK parameter is detected at a high or low level. Then, a decision can be based on another PK parameter, and so forth. The method is data-based and can be used for predicting the relationship between PK parameter edema and pain levels. Nearest neighbor algorithms are distance based classification methods that are used to assign the closest match to an individual and are useful for individual-to-individual comparison of complex components.

Principal components, factor analysis, multidimensional scaling and other methods of data reduction are methods to reduce the number of combinations of data points for an effective classification. Likelihood models are methods using statistical data and probability models to provide optimal use of statistical information, where applicable. Likelihood models provide a specific description of the pattern of variation in data and can be used for estimation and hypothesis testing. Hypothesis testing is a formal process of using data to make decisions. Hypothesis testing can be used to test whether a molecule or set of molecules is useful and should be included in a group. Hypothesis testing can also be used to decide if a pool of individuals is significantly different from another pool or group of individuals.

Derived variables can be created and used to increase dimensionality beyond the starting parameters in order to help a statistical method achieve an effective classification. For example, interaction terms formed by multiplying some or all of the parameters pairs. “Kernel density” estimation and other smoothing techniques are methods used for the purpose of averaging out or eliminating noise in data or statistical variation in data.

Bootstrap and other statistical resampling techniques are methods used to resample from the data in order to assess the variability of the system computed from such data. “Artificial intelligence,” including artificial neural networks, machine learning, data mining, and boosting algorithms can also be used. An artificial neural network is a computational method trained on a training set to make a new classification, for example, a training set of molecules in a reference population to classify a new individual. “Machine learning” is a collection of automated methods in which training can be used to learn what distinguishes a group, for example, groups of different health states, and is then used to classify an individual into a group. A boosting algorithm is an example of machine learning and is based on taking a simple system of classification methods to assemble more complex methods. For example, in a boosting algorithm, the expression levels of molecules taken one at a time can be analyzed in a particular sequence to generate a more effective method. Data mining is a method based on learning and inferring from large bodies of data and is useful for understanding how to use a large data set for calculations.

The invention provides statistical calculations for the degree of confidence associated with the assignment of an individual to a health state. Accordingly, two individuals can both have multidimensional coordinate points within a particular health-associated reference expression region, for example, a region corresponding to reference individuals having cancer, but with different levels of confidence for the diagnosis, for example, one individual can have a 98% confidence of the diagnosis while the other individual has an 85% confidence.

As used herein, “designing” refers to changes in the active agent's dose, formulation and/or regiment based on the patient data, using computerized determining, logic and the experience of one of ordinary skill in the art. Said designing may be done in accordance with the invention aided by statistical and data-mining techniques known to those of ordinary skill in the relevant art that can identify the pharmacokinetic parameters and formulation components of greatest importance. It will be appreciated by those of ordinary skill that iterative process claimed herein will lead to steady state PK profiles for the NSAID and the anti-hypertensive within 4-6 cycles of dose changes and single dose and steady state predetermined ranges will be used as appropriate.

Complete pharmacokinetic profiles may result in such a large number of variable parameters that there would be too many pairwise correlations between the variables to reasonably consider. In particular, with so many parameters graphical display, usually a powerful way to begin data analysis, may not be useful. Surprisingly, with 12 variables, there will be more than 200 three-dimensional scatterplots. To put the such complex data in a more useful form, it is necessary to reduce the number of variables to a few, interpretable linear combinations which account for most of the variability in the data.

“Factor Analysis” offers a way to reduce the data to a few critical parameters. In every factor analysis, there are the same number of factors as there are variables. Each factor captures a certain amount of the overall variance and the factors are always listed in the results in the order of how much of the overall variance they explain. (see U.S. Pat. No. 8,660,370.)

“Principal components analysis is another data reduction method. Suppose that there are a dozen variables, principal components analysis can reduce these to a few (2-4) principal components. Principal components analysis, like factor analysis, can be performed on raw data or on a correlation or a covariance matrix. (see U.S. Pat. No. 7,412,333.).

Data reduction can also be done with clustering, stepwise variable selection and factor analysis techniques to identify the most parsimonious set of data that needs to be collected. Internal (bootstrap) validation and comparisons with external data sources will be used to validate selected variables.

In another approach to data analysis, U.S. Pat. No. 8,999,648 is directed to system for classifying a biological sample from a cancer patient and calls for at least one statistical classification program such as of k-nearest neighbors (kNN), linear discriminant analysis, principal components analysis, nearest centroid classification, and support vector machines.

As used herein, “determining the level of efficacy” refers to the use of objective (e.g., pharmacokinetic) and subjective tests (e.g., pharmacodynamic), signs and symptoms to characterize, quantify or evaluate how well symptoms (e.g., pain) are controlled by the administration of the active ingredient (e.g., celecoxib and a anti-hypertensive).

As used herein, “determining the level of toxicity” refers to the use of objective and subjective tests, signs and symptoms to characterize, quantify or evaluate the significance of any side effects produced by the administration of the active ingredient.

As used herein, “pain control is adequate” refers to a level of pain the patient is willing to live with and which does not significantly impair the patient's functioning in society or the patient's physiologic functions.

As used herein, “toxicity is acceptable” refers to the absence of significant side effects and a level of toxicity that the patient is willing to live with and does not significantly impair the patient's functioning in society or the patient's physiologic functions.

As used herein, “trace edema” is edema that is just above the threshold for detection on physical exam (inconsistently pitting) and does not significantly impair the patient's functioning in society or the patient's physiologic functions.

As used herein, “NSAID” or “non-steroidal anti-inflammatory drug” refers to a class of drugs which provide pain-reducing and fever reducing effects as well as anti-inflammatory effects in a subject, e.g., a human patient. NSAIDs include both COX-1 and COX-2 inhibitors.

As used herein, a “COX-1 inhibitor” refers to a non-steroidal anti-inflammatory drug that is capable of directly targeting the COX-1 enzyme in a subject and inhibits at least some COX-1 activity, e.g., aspirin.

As used herein, a “COX-2 inhibitor” refers to a non-steroidal anti-inflammatory drug that is capable of directly targeting the COX-2 enzyme in a subject and inhibits at least some COX-1 activity, e.g., celecoxib. As used herein, a “mixed COX-1 and COX-2 inhibitor” refers to a non-steroidal anti-inflammatory drug that is capable of directly targeting both the COX-1 and COX-2 enzymes in a subject and inhibits at least some COX-1 and COX-2 activity, e.g., ibuprofen.

As used herein the term “anti-hypertensive” refers to any substance that promotes the reduction of blood pressure. Suitable anti-hypertensives for use in the compositions and methods disclosed herein include but are not limited to azilsartan, candesartan, eprosartan, irbesartan, losartan, olmesartan, telmisartan, valsartan, benazepril, captopril, enalapril, fosinopril, lisinopril, moexipril, perindopril, quinapril, ramipril, trandolapril, and combinations thereof.

As used herein “controlled release” refers to the delivery of the NSAID, the anti-hypertensive, or both in response to in vivo stimuli. For example, pH changes in the digestive tract. As used herein “delayed release” refers to prolonged dissolution time, e.g., increase dissolution time by 2, 4 or 6 hours. (see e.g., U.S. Pat. No. 8,992,979)

As used herein “enteric coated” refers to a dosage form with polymer barrier which is resistant to dissolution at gastric pH levels but dissolves at the higher pH levels typical of in the intestine, applied to the composition comprising a NSAID and an anti-hypertensive.

As used here the combination can also include a “fixed dose combination” (FDC) or simply dosing with multiple pills each of a single agent to achieve a desired effect.

The invention provides compositions and methods for individualized therapy of pain, including but not limited to arthritic pain, using a non-steroidal anti-inflammatory drug (NSAID), preferably celecoxib in combination with an anti-hypertensive, preferably an angiotensin receptor blocker (“ARB”) or an angiotensin converting enzyme inhibitor (“ACE”) inhibitor. In addition, the invention provides methods for predicting the outcome of the therapy of pain with a composition comprising NSAID, preferably celecoxib, and an anti-hypertensive, preferably ARB or ACE inhibitor. Further, the invention provides methods of using a NSAID, preferably celecoxib, and an anti-hypertensive, preferably ARB or ACE inhibitor, in the manufacture of medicament for the treatment of pain.

The invention provides endpoints (i.e., an individualized drug therapy) based on the achievement of predetermined PK results, as well as the clinical condition of the patient.

In one embodiment, provided is a composition for treating pain without inducing edema comprising a NSAID and a anti-hypertensive, wherein the composition is administered in a fixed-dose combination. Any type of pain may be treated by the composition, including arthritic and osteoarthritic pain.

The NSAID may be one or more of the following NSAIDs, but is not limited thereto: diclofenac, diflusnisal, etodolac, fenoprofen, flubiprofen, ibuprofen, indomethacin, ketoprofen, ketorolac, meclofenamate, nabumetone, naproxen, oxaprozin, phenylbutazone, piroxicam, salicylate, sulindac, tolmetin, celecoxib, rofecoxib, etoricoxib, lumiracoxib, parecoxib, pomacoxib, valdecoxib, chlorthalidone and combinations thereof. In a preferred embodiment, the COX-2 inhibitor is celecoxib.

In a preferred embodiment, the anti-hypertensive is an ARB or an ACE inhibitor.

Angiotensin II Receptor blockers, include but are not limited to, azilsartan, candesartan, eprosartan, irbesartan, losartan, olmesartan, telmisartan, valsartan and combinations thereof.

ACE (Angiotensin Converting Enzyme) Inhibitors include, but are not limited to, benazepril, captopril, enalapril, fosinopril, lisinopril, moexipril, perindopril, quinapril, ramipril, trandolapril, and combinations thereof.

The composition may be in the form of a capsule, a pill, a syrup, a controlled release device, or an injectable solution, and the release of one or both of the NSAID and the anti-hypertensive may be controlled. The NSAID and the anti-hypertensive may be released substantially simultaneously, or one may be released before the other, in any order. The composition may be administered, for example, daily, twice a day, three times a day, four times a day, or every other day.

One or more pharmaceutically acceptable carriers or excipients may be included in the composition. Any suitable pharmaceutical carrier can be used in accordance with the invention. Suitable pharmaceutical carriers include, without limitation, sterile water; saline, dextrose; dextrose in water or saline; hydroxypropylmethylcellulose, ethylcellulose, or other suitable cellulose derivatives, povidone, acrylic and methacrylic acid co-polymers, pharmaceutical glaze, gums, solvents, ethanol, isopropyl alcohol, methylene chloride or sugar, lactose, gelatin, starch, silicon dioxide, diethyl phthalate, diethyl sebacate, triethyl citrate, cronotic acid, propylene glycol, butyl phthalate, dibutyl sebacate, castor oil, diethyl phthalate, diethyl sebacate, lactose, dextrose, saccharose, cellulose, starch or calcium phosphate, olive oil or ethyl oleate silica, talc, stearic acid, magnesium or calcium stearate and/or polyethylene glycols; clays, gum tragacanth or sodium alginate, binding agents such as starches, arabic gums, gelatin, methylcellulose, carboxymethylcellulose, polyvinylpyrrolidone, alginic acid, sodium starch glycolate, polysorbates, laurylsulphates; and other therapeutically acceptable accessory ingredients, such as humectants, preservatives, buffers and antioxidants, which are known additives for such formulations, lactose, dextrose, saccharose, cellulose, ethyl oleate, talc, stearic acid, magnesium or calcium stearate and/or polyethylene glycols, arabic gums, gelatin, methylcellulose, carboxymethylcellulose, polysorbates and combinations thereof.

It should be understood that in addition to the ingredients particularly mentioned above, the formulations of this invention can include other suitable agents such as flavoring agents, preservatives and antioxidants. Such antioxidants would be food acceptable and could include vitamin E, carotene, BHT or other antioxidants known to those of skill in the art. In embodiments where the composition is in the form of a pill, the pill may be bilayered, enteric coated, or a combination thereof. The pill may be administered orally.

The composition may be administered in a fixed dose combination of the NSAID and an antihypertensive selected from ARBs or ACE inhibitors.

The composition may include any suitable NSAID and anti-hypertensive dosage. For example, the composition may comprise 50 to 400 mg, 75 to 350 mg, 100 to 300 mg, 150 to 250 mg, 50 mg, 100 mg, 200 mg, or 400 mg of NSAID. The composition may comprise 2.5 to 200 mg, 10 to 150 mg, 20 to 100 mg, 40 mg, 100 mg, 150 mg, or 200 mg of anti-hypertensive

The composition may be administered to any suitable subject, including mammals. Suitable mammals include but are not limited to humans.

The PK parameter determined/used in the methods of the invention is one or more of concentration, concentration time course, peak concentration, time after administration to peak concentration, terminal half-life, AUC, bioavailability, absorption, volume of distribution, metabolism, excretion, biotransformation, clearance or a combination thereof.

Any suitable NSAID can be used in accordance with the invention, including without limitation, a COX-1-specific inhibitor, a COX-2-specific inhibitor, a mixed COX-1 and 2 inhibitor or a combination thereof. As such, the NSAID can be a salicylate, propionic acid derivative, acetic acid derivative, enolic acid (oxicam) derivative, anthranilic acid derivative (fenamatea) or combinations thereof. Accordingly, the NSAID can be, aspirin (acetylsalicylic acid), ibuprofen, naproxen, indomethacin, sulindac, piroxicam, clonixin, preferably celecoxib or a combination thereof. In addition, the invention can be used with combinations of NSAIDs and other analgesic drugs such as lidocaine, opiates, acetaminophen, tricylic antidepressants, anticonvulsants, carbamazepine, gabapentin, and pregabalin; other anti-inflammatory drugs such as steroids and immunosuppressants. Further, the invention can be used with combinations of NSAIDs and other therapies for arthritis, including but not limited to, methotrexate and gold-salts.

The composition comprising a NSAID and an anti-hypertensive can be administered in accordance with the invention via any suitable route including, without limiting, orally, rectally, by inhalation, trans-cutaneously, by injection, intra-venously or intra-arterially. The non-NSAID component of any combination therapy can be administered in accordance with the invention by any suitable route including, without limiting, orally, rectally, by inhalation, trans-cutaneously, by injection, intra-venously or intra-arterially. Any suitable regimen can be used in accordance with the invention to administer two or more drug components, including without limitation, simultaneously (within minutes of one another), substantially simultaneously (within an hour of one another) or at different times.

Other treatments for chronic diseases can be included such as treatments for diabetes, cardiovascular diseases, dementia, cholesterol, and hypertension. For example, fixed dose combination for pain relief without edema, may be practiced in conjunction with the administration of a prescribed cholesterol regulator, such as atorvastatin.

Any suitable PK parameter or parameters can be used in accordance with the invention, including without limiting concentration, concentration time course, peak concentration, and time after administration to peak concentration, terminal half-life, AUC, bioavailability, absorption, distribution, metabolism, excretion, biotransformation, or a combination thereof.

Any suitable pharmacodynamic parameter or parameters can be used in accordance with the invention, including without limiting the physiological changes of cells, tissues and ligaments of a patient, patient or physician reported pain level, the frequency of side effects, or a combination thereof.

Any suitable method for the assessment of pain known to those of ordinary skill in the art can be used in accordance with the invention, including, but not limited to, one-dimensional pain intensity scales, Wisconsin Brief Pain Questionnaire, Brief Pain Inventory, The McGill Pain Questionnaire and the short-form, McGill Pain Questionnaire (See Breivik et al.: Assessment of pain, British Journal of Anaesthesia 2008, 101 (1): 17-24).

The following examples further illustrate the invention but, of course, should not be construed as in any way limiting its scope.

### Example 1

The approved prescribing information for Celebrex® (celecosib) as listed on its package insert for US/EU/ROW instructs that a physician should use lowest effective dose for the shortest duration consistent with treatment goals for the individual patient. For four of the six approved indications the package insert includes a 100 mg BID regimen:

1) Osteoarthritis (OA): 200 mg QD or 100 mg BID

2) Rheumatoid Arthritis (RA): 100 mg BID or 200 mg BID

3) Juvenile Rheumatoid Arthritis (JRA): 50 mg BID in patients 10-25 kg. 100 mg BID in patients more than 25 kg

4) Ankylosing Spondylitis (AS): 200 mg once daily single dose or 100 mg BID.

5) Acute Pain (AP) and 5) Primary Dysmenorrhea (PD). 400 mg initially, followed by 200 mg dose if needed on first day. On subsequent days, 200 mg BID as needed.

Unexpectedly, however, the inventor's analysis of the actual prescribing behavior using Evaluate Pharma/IMS database determined that the 200 mg is the predominant dose being prescribed by physicians by more than 10 to 1. These data are consistent with data from a MEPS survey (Table 1) and Medicaid survey (Table 2). In view of predominance of the 200 mg dosage form sales and the evidence that the 100 mg and 200 mg doses produce overlapping PK and pharmacodynamic results, it is questionable that the package insert's admonition that “the lowest dose of Celebrex® should be sought for each patient” is followed. Instead, the data indicates that it is likely that there are numerous patients at risk for ADRs because their celecoxib dose is higher than it needs to be (i.e., e.g., 200 mg BID rather than 100 mg BID).

TABLE 1
MEPS Survey Data

| | Strength | USA sales | USA sales |
|---|---|---|---|
| Package description | (mg) | 2008 ($m) | 2011 ($m) |
| 100 capsule in bottle (0025-1525-31) | 200 | 1,553 | 1,650 |
| 500 capsule in 1 bottle (0025-1525-51) | 200 | 323 | 132 |
| 100 capsule in 1 bottle (0025-1520-31) | 100 | 65 | 142 |
| 500 capsule in 1 bottle (0025-1520-51) | 100 | — | 17 |
| 100 blister pack in 1 carton (0025-1520- | 100 | — | 7 |
| 34) >1 capsule in 1 blister pack | | | |
| 120 capsule in 1 bottle (63629-3021-5) | 200 | — | 2 |
| 30 capsule in 1 bottle, plastic (67544- | 200 | — | 32 |
| 204-30) | | | |
| Total | | 1,989 | 1,982 |

TABLE 2
Prescribing information derived from Medicaid

| Dose | Number of | Number of | Number of | Number of | Number of | Number of | Number of |
|---|---|---|---|---|---|---|---|
| (mg) | RXs 2008 | RXs 2009 | RXs 2010 | RXs 2011 | RXs 2012 | RXs 2013 | RXs 2014 |
| 50 | 293 | 485 | 856 | 1169 | 1167 | 1396 | 726 |
| 100 | 33,436 | 36,023 | 43,755 | 47,524 | 35,399 | 34,178 | 16,628 |
| 200 | 320,628 | 330,521 | 380,546 | 384,404 | 285,764 | 250,985 | 114,532 |
| 400 | 1,383 | 1,636 | 3,116 | 3,476 | 2,565 | 2,240 | 1,093 |
| Total | 355,740 | 368,665 | 428,273 | 436,573 | 324,895 | 288,799 | 132,979 |

### Example 2

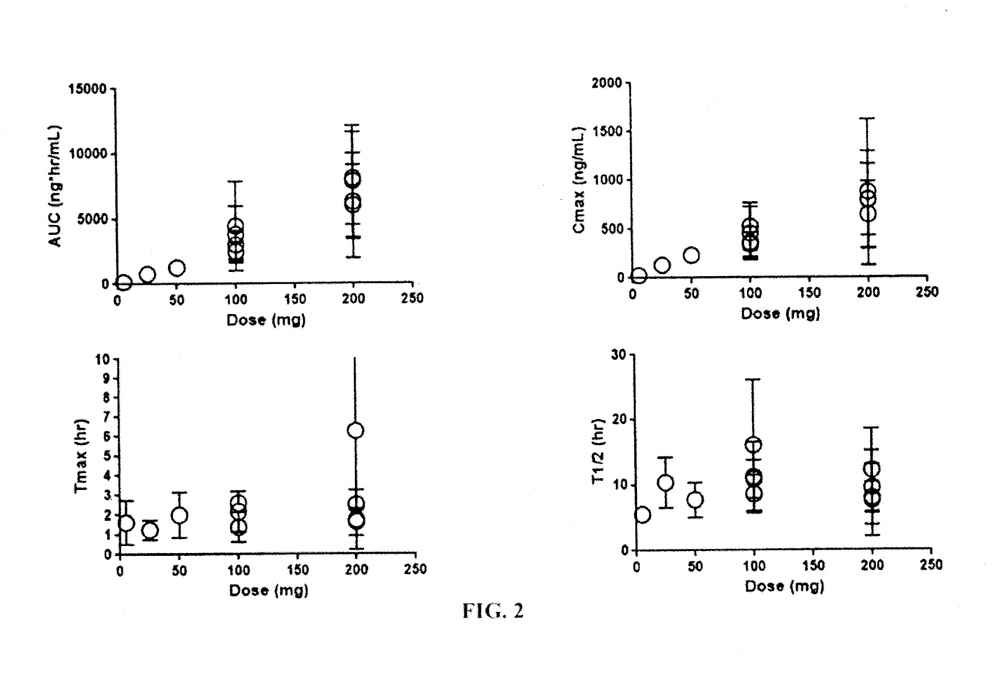

The combined plots of published pharmacokinetic data including those from the Summary basis for approval are shown in FIG. 2. The variability of Celebrex® pharmacokinetics were unexpectedly high. The PK results for the 200 mg dose shows a substantial overlap with that of the 100 mg dose. Accordingly, the dose proportionality may not be as is described by the package insert for Celebrex®. As a result of the failure to determine and pursue target PK ranges, in some instances patients receiving 100 mg patients may not get enough of the drug and the 200 mg patients may receive too much of the drug.

### Example 3

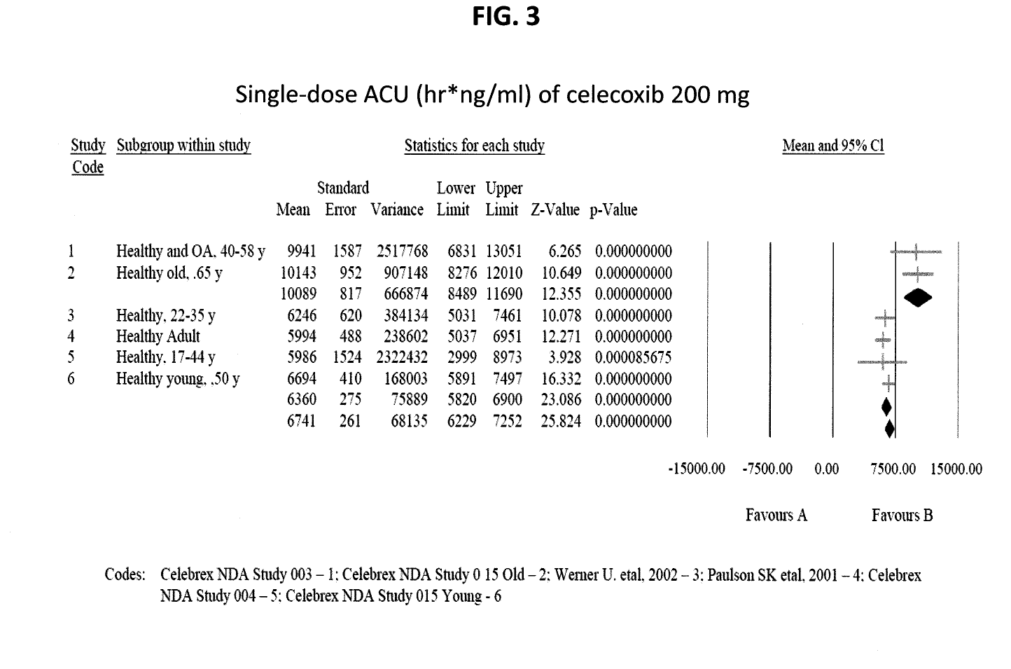

Applicant's meta analysis of the reported PK parameters in different populations demonstrates that the elderly show a higher variability than younger patients. For Example, when the applicant's meta analysis is presented in age-based subgroups, the elderly and younger patients demonstrate highly significant differences in drug exposure as defined by AUC (FIG. 3). In other words, the most efficacious celecoxib dosage is not well defined among the elderly. The problem may be more widespread than expected as elderly here is defined as patients greater than >40 or >50, not the usually definition of elderly (age greater >65). Previously, there has been reported impaired PK with elderly and the package insert issued warning on impaired PK in elderly but did not suggest dose reduction. Our finding suggests that the issue is more substantial and more widespread and includes middle aged groups also.

There is variability in PK results within groups and the Cₘₐₓ and AUC overlap between the 100 mg and 200 mg groups indicates that correctly dosing elderly patients to maximize celecoxib efficacy at the lowest doses possible depends on many individualized, unpredictable variables. Based on the wide range of AUC values, some patients receiving 100 mg may not get enough of the drug and the 200 mg patients may receive too much of the drug (FIG. 2).

### Example 4

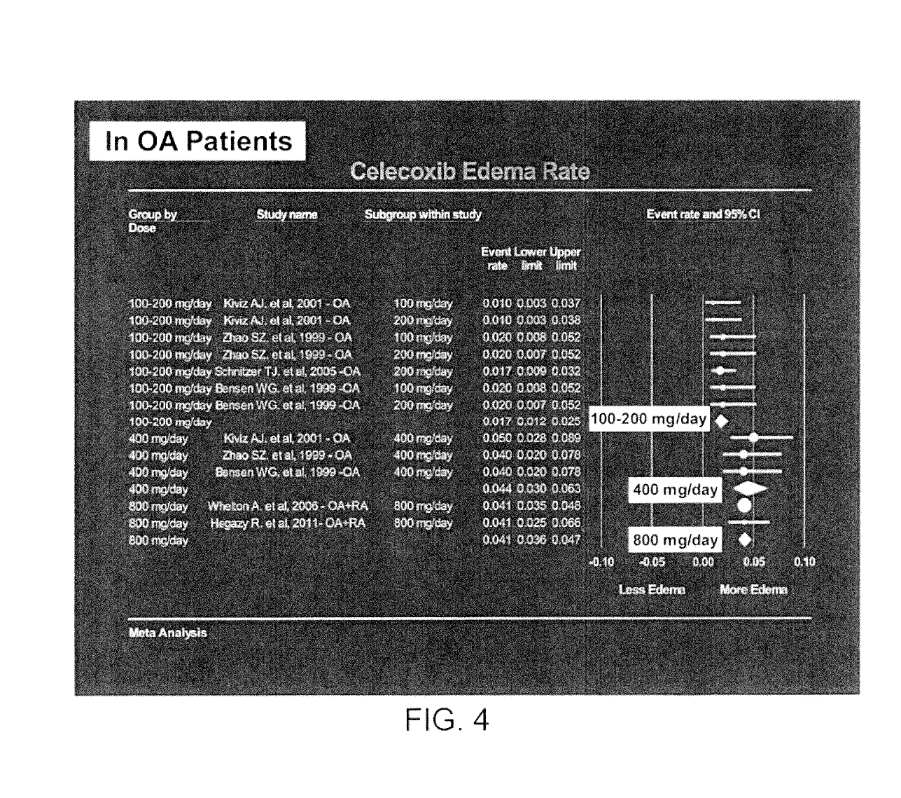

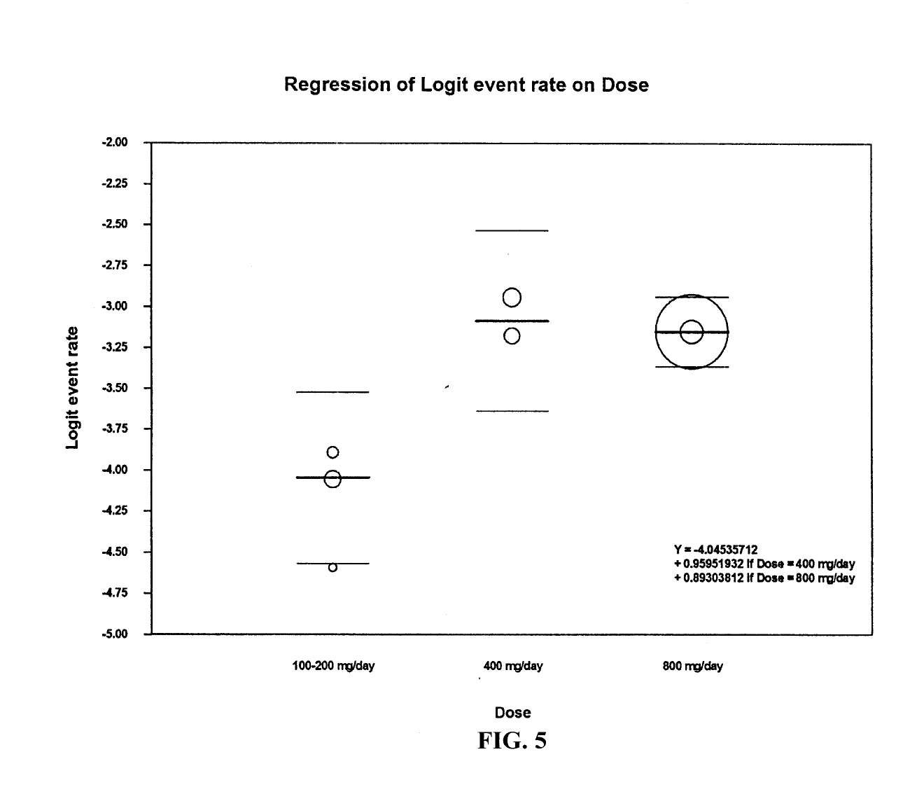

Applicant's meta analysis of the reported edema rates in different osteoarthritic populations receiving doses of celecoxib ranging from 100 mg/day to 800 mg/day reveals that edema event rates are significantly higher in osteoarthritic populations receiving doses of celecoxib that are greater than 200 mg/day. For Example, both the upper limit of the edema event rate and the average edema event rate in osteoarthritic populations receiving a 400 mg/day dose of celecoxib can be more than twice as high as the upper limit edema event rate and the average edema event rate seen in osteoarthritic populations receiving a 100 or 200 mg/day dose of celecoxib (FIGS. 4-5). Additionally, Applicant's meta analysis of the reported edema rates in different osteoarthritic populations receiving doses of celecoxib ranging from 100 mg/day to 800 mg/day reveals that patients receiving a 100 or 200 mg/day dose of celecoxib experience remarkably similar edema event rates. This analysis indicates that a patient who is selected to receive a 200 mg/day dose of celecoxib based on, for example, their individual pharmacokinetic data using one or more of the methods described herein, will not be at a higher risk for an edema event than a patient receiving a 100 mg/day dose of celecoxib, and vice versa.

### Example 5

Applicant has also compared the edema in patient populations receiving celecoxib alone, celecoxib in combination with a variety of antihypertensives, including thiazides, celecoxib in combination with hydrochlorothiazide, celecoxib with a non-thiazide diuretic and no anti-hypertensives, and celecoxib with a non-thiazide anti-hypertensive. To support this study a database was created which contains: 1) Claims data from Symphony pertaining to anti-hypertensives, Statins, COX-2's, and NSAIDS. The data span the most recent 36 months and 2) registry data from the ACC reporting Blood Pressure (systolic/diastolic), peripheral edema flags (yes, no, missing), Heart rate, LDL, Glucose Level, Ejection fraction, GFR, Height, Weight, BMI, and the like.

Symphony Database® contains true patient level data—All Data Sources be it RX or MX claims is tied back to individual patients which is tracked and then encrypted based on first name, last name, gender, DOB and zip code to give an accurate picture of patient level informatics year over year regardless of insurance changes. The source of Managed Markets Rx claims data comes from various providers, including Intelligent network services (Switch Data) as well as direct data feeds from pharmacies that do not use Switches so it does not create payer biases.

The properties for the Symphony Database® are: 1) Takes Celebrex, AH, Statin or NSAID or have OA, RA or some other form of Arthritis, 36 months, 2) Time Frame=Jan. 1, 2012-Dec. 31, 2014 (3 years), 3) Number of files=201, 4) Size=561 GB zipped (˜2.5 TB), 5) Unique Patients=162 million, 6) Patients on Celebrex=4.3 million, 7) Patients that have OA=16.3 million (15.4 million only OA), 8) Patients that have RA=2.3 million (1.4 million only RA).

The properties of the ACC registry are: 1) Have 3+BP readings, 2) Time Frame=Jan. 1, 2012-Dec. 31, 2014 (3 years), 3) Number of files=2, 4) Size=590 MB, 51 MB, 5) Unique Patients=1.58 million, 6) Patients with BP readings=1.58 million, 7) Patients with Edema Flag True=870K.

The edema rate was then measured in the aforementioned database. The incidence of edema was higher for OA patients than RA, other Arthritis, or Arthritis free patients. The incidence of edema increased when patients was taken Celebrex for all groups except for RA and no Arthritis free patients. Overall OA seems to be susceptible to Celebrex induced edema. The results confirmed the meta analysis shown above.

TABLE 3
Shows cases of Edema (%) among OA, RA,
Other Arthritis, and No Arthritis.

| | | | Other | No |
|---|---|---|---|---|
| Group | OA | RA | Arthritis | Arthritis |
| W/o | 12,150 (4.7%) | 3,337 (3.0%) | 164,837 (2.3%) | 69 (0.4%) |
| Celebrex | | | | |
| W/ | 254 (6.0%) | 82 (3.3%) | 1702 (4.1%) | 451 (0.4%) |
| Celebrex | | | | |

### Example 6

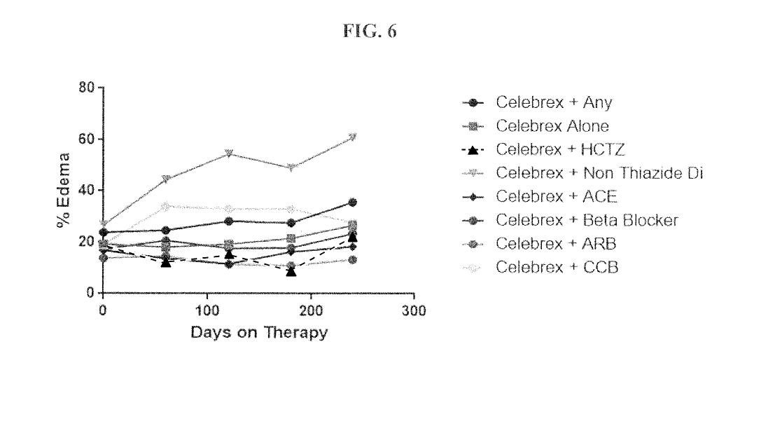

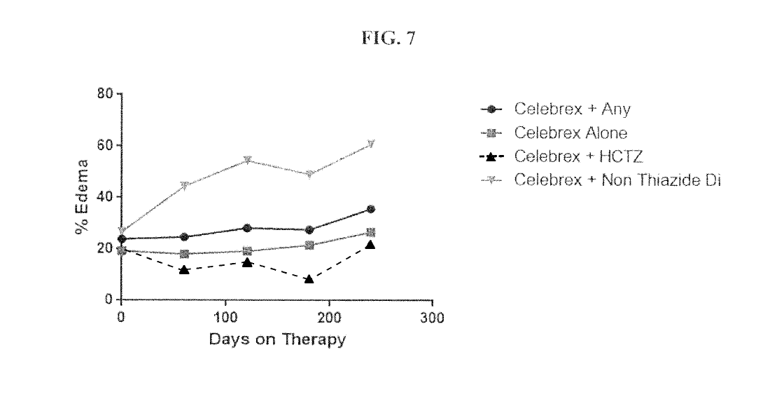

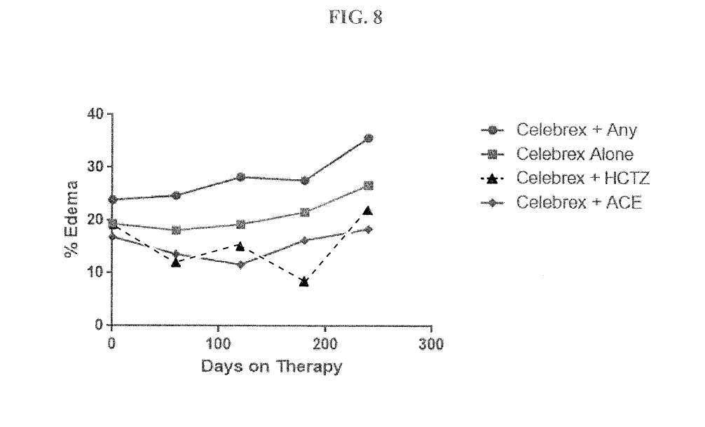

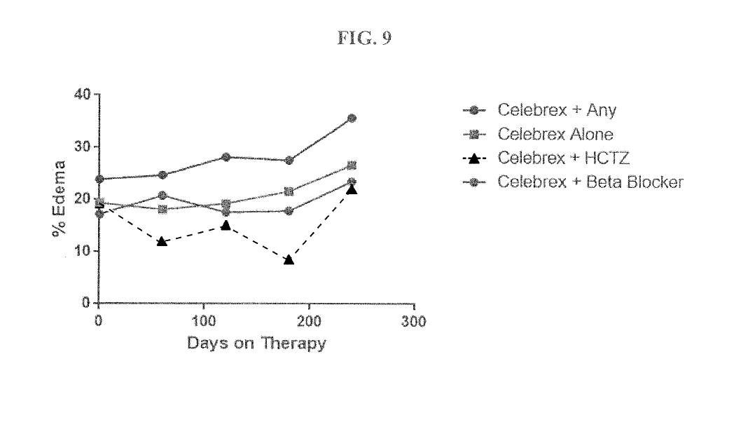

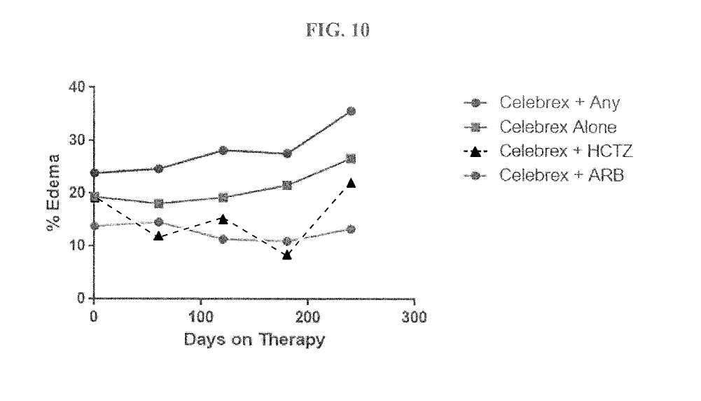

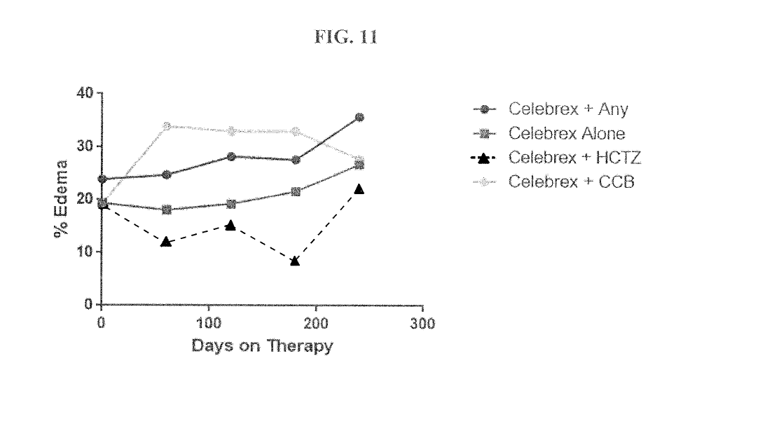

The same database used in Example 5 was used to determine whether a specific anti-hypertensive would more effectively block the development of edema by patients taking Celebrex. There was a steady increase in incidence of edema among patient taking Celebrex only (FIGS. 6-10). This trend was exacerbated by the addition of other drugs (FIGS. 6-10). The incidence of edema was lower for patients taking Celebrex and an anti-hypertensive drug (FIGS. 6-10). The order of effectiveness of the classes of anti-hypertendive drugs in preventing edema was surprisingly ARB >>ACE inhibitor=HCTZ (FIGS. 6-10). The Beta blockers were ineffective (FIGS. 6 & 9). The CCB (FIGS. 6 & 11) and non HCTZ thiazides (FIG. 11) aggravated the edema.

### Example 7

The same database in Example 5 was used to determine whether the thiazide diuretic hydrochlorothiazide (HCTZ) would reduce the incidence of edema in patients taking Celebrex®. There was a steady increase in incidence of edema among patient taking Celebrex® only. This trend was exacerbated by additional Rx. The incidence was lower for patients taking Celebrex® and HCTZ. In contrast, the incidence of patients taking Celebrex® and non-thiazide diuretics resulted in more than doubling of the incidence of edema. The data is surprising in that edema can only be controlled selectively by HCTZ (a thiazide diuretic) and not by other non-thiazide diuretics. The non-thiazide diuretics include: 1) Loop: torsemide, furosemide, bumetanide, ethancrynic acid, 2) Carbonic Anhydrase Inhibitors: acetazolamide, dichlorphenamide, methazolamide, 3) Potassium sparring: triamterene, spironolactone, amiloride, and 4) Others: pamabrom, mannitol.

TABLE 4
presents cases of Edema (%) among patients taking Celebrex and any other
Rx, Celebrex only, Celebrex + HCTZ, and Celebrex + non-thiazide diuretics.

| | | | | Celebrex + |
|---|---|---|---|---|
| Days of | Celebrex + | Celebrex | Celebrex + | non-Thiazide |
| Therapy | Any Rx | Only | HCTZ | Diuretics |
| <60 | 139,389 (23.9%) | 20,663 (19.4%) | 455 (19.1%) | 2146 (26.7%) |
| 60-120 | 14,361 (24.7%) | 913 (18.1%) | 50 (12.0%) | 160 (44.4%) |
| 120-180 | 12,272 (28.2%) | 678 (19.3%) | 33 (15.2%) | 125 (54.4%) |
| 180-240 | 12,620 (27.6%) | 661 (21.6%) | 47 (8.5%) | 49 (49.0%) |
| >240 | 55,396 (35.7%) | 1,991 (26.7%) | 77 (22.1%) | 453 (60.9%) |

### Example 8

The frequency of patients experiencing edema was examined. Two groups were used—those taking celebrex for any length of time and those taking celebrex for more than 180 days. As shown below—those taking celebrex for greater than 180 days experienced higher incidence of edema. In both cases, combining celebrex with either ACE or ARB effectively inhibited celebrex induced edema significantly. This was more evident when comparing the celebrex+ARB/ACE inhibitor versus Celebrex+any drug regardless of class. The results are surprising as addition of another drug to celebrex regime would be expected to induced additional drug induced toxicitiy. Indeed addition of non-thiazide anti-hypertensive to Celebrex significantly worsen the incidence of edema.

Table 5 showing incidence of edema when patients taking Celebrex alone, Celebrex plus any other drug, Celebrex and various classes of anti-hypertensive. For this group, the patients are on celebrex for any length of time.

TABLE 5
Incidence of Edena, all patients on celecoxib

| #Pts | Edema | No Edema | Total Pts # | % Edema |
|---|---|---|---|---|
| Alone | 4,975 | 19,931 | 24,906 | 20% |
| Any Drug | 63,550 | 170,488 | 234,038 | 27% |
| HCTZ | 119 | 543 | 662 | 18% |
| NONTHIAZ | 1,018 | 1,948 | 2,966 | 34% |
| ACE | 321 | 1,602 | 1,923 | 17% |
| ARB | 213 | 1,363 | 1,576 | 14% |
| BETA | 1,771 | 7,712 | 9,483 | 19% |
| CCB | 460 | 1,541 | 2,001 | 23% |
| Totals | 72,427 | 205,128 | 277,555 | |

Table 6 presents the incidence of edema when patients taking Celebrex alone, Celebrex+any other drug, Celebrex+various classes of anti-hyperensive. For this group, the patients are on celebrex for more than 180 days.

TABLE 6
Incidence of Edema, patients on celecoxib more than 180 days.

| #Pts | Edema | No Edema | Total Pts # | % Edema |
|---|---|---|---|---|
| Alone | 4975 | 19931 | 24906 | 20% |
| Any Drug | 23264 | 44752 | 68016 | 34% |
| HCTZ | 21 | 103 | 124 | 17% |
| NONTHIAZ | 307 | 228 | 535 | 57% |
| ACE | 59 | 272 | 331 | 18% |
| ARB | 37 | 254 | 291 | 13% |
| BETA | 491 | 1708 | 2199 | 22% |
| CCB | 126 | 306 | 432 | 29% |
| Totals | 29280 | 67554 | 96834 | |

### Example 9

The following is a prophetic example of how those of ordinary skill can predetermine PK parameters and their ranges in accordance with the invention.

The serum or plasma concentrations of the NASID and antihypertensive could be determined for, e.g., 20 responders and 20 nonresponders to NASID/antihypertensive regimen in accordance with the invention, at each hour for 12 hours. PK parameters would then be determined from the concentrations/time point data resulting in, e.g., 12 variable PK parameters. Factor Analysis could then be used to reduce the data to the PK parameters (e.g., 2-3) which are associated with most of the variability in the data. The importance of the identified PK parameters could be confirmed by another data reduction method, e.g., principal components analysis. Data reduction could be followed by cluster analysis (e.g. K-means analysis), using the PK profiles of responders and nonresponders to identify what ranges of values for said PK parameters are predictive of response to therapy.

Alternatively, all of the PK parameters obtained from training PK profiles of known outcome to establish clusters of PK data profiles for responders and non responders which are at a statistically significant distance from one another in multidimensional space (with each PK parameter constituting one dimension). New PK profiles could then be mapped in this multidimensional space and their map location compared to the clusters for the training PK profiles.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim the following:

1. A composition for treating pain without inducing edema comprising a NSAID and an anti-hypertensive in a fixed-dose combination and, optionally, further comprising one or more pharmaceutically acceptable carriers.

2. The composition of claim 1, wherein the NSAID is aspirin, diclofenac, diflusnisal, etodolac, fenoprofen, flubiprofen, ibuprofen, indomethacin, ketoprofen, ketorolac, meclofenamate, nabumetone, naproxen, oxaprozin, phenylbutazone, piroxicam, salicylate, sulindac, tolmetin, celecoxib, rofecoxib, etoricoxib, lumiracoxib, parecoxib, polmacoxib, valdecoxib, chlorthalidone, or a combination thereof.

3. The composition of claim 2, wherein the NSAID is celecoxib, etoricoxib, lumiracoxib, parecoxib, polmacoxib, valdecoxib, chlorthalidone, or a combination thereof.

4. The composition of claim 3, wherein the NSAID is celecoxib.

5. The composition of claim 1, wherein the anti-hypertensive is azilsartan, candesartan, eprosartan, irbesartan, losartan, olmesartan, telmisartan, valsartan, benazepril, captopril, enalapril, fosinopril, lisinopril, moexipril, perindopril, quinapril, ramipril, trandolapril, or a combination thereof.

6. The composition of claim 5, wherein the anti-hypertensive is olmesartan.

7. The composition of claim 1, wherein the composition is in the form of a capsule, a pill, a syrup, a controlled release device, or an injectable solution.

8. The composition of claim 7, wherein the release of one or both of the NSAID and the anti-hypertensive is controlled.

9. The composition of claim 8, wherein the composition is in the form of pill.

10. The composition of claim 1, wherein the fixed dose combination comprises a ratio of mg NSAID to mg anti-hypertensive from 20:1 to 4:1.

11. The composition of claim 1, wherein the NSAID is celecoxib and the fixed dose combination comprises 50 to 400 mg of celecoxib.

12. The composition of claim 1, wherein the fixed dose combination comprises an angiotensin receptor blocker or an angiotensin converting enzyme inhibitor, and the fixed dose combination comprises 1 to 100 mg of olmesartan or 1 to 100 mg of lisinopril.

13. A method for individualized therapy of arthritic pain using a COX-2 inhibitor, comprising:
a. administering, or causing to be administered, a first COX-2 inhibitor formulation comprising a first dose of a COX-2 inhibitor combined with an anti-hypertensive in specific amounts, to a first patient suffering from pain;
b. determining, or causing to be determined, the COX-2 inhibitor and anti-hypertensive concentrations in either of the first patient's serum or plasma at a plurality of time data points after the first COX-2 inhibitor formulation has been administered to the first patient;
c. transforming, or causing to be transformed, the first patient's COX-2 and anti-hypertensive concentration/time data points into one or more pharmacokinetic (PK) parameters;
d. if the pain control is adequate and edema is tolerable, comparing the first patient's PK parameters to predetermined PK parameters to determine if the first patient's PK parameters are within predetermined ranges of values for each of the predetermined PK parameter and if one or more of the first patient's PK parameters fall outside of its predetermined range, designing a new COX-2 inhibitor formulation, wherein the dose of said COX-2 inhibitor, the anti-hypertensive, or both is different from that of the first COX-2 inhibitor formulation;
e. administering, or causing to be administered, the new COX-2 formulation to the first patient;
f. repeating steps b-e until all the PK parameters used in step d are within said predetermined ranges, and
g. if pain control is adequate and edema is tolerable, maintaining the first patient on the COX-2 inhibitor formulation at frequency of administration that satisfied the comparison with predetermined ranges in step d.

14. The method of claim 13, wherein the COX-2 inhibitor formulation is administered orally.

15. The method of claim 13, wherein said predetermined PK ranges are based the PK parameters of other patients suffering from pain who had been treated with said COX-2 inhibitor formulation such that their pain control is adequate, edema is tolerable, wherein the PK parameters for the comparisons were identified using either a data reduction protocol or clustering and wherein the difference in the identified clusters is statistically significant.

16. The method of claim 13, wherein the COX-2 inhibitor is celecoxib.

17. The method of claim 13, wherein the PK parameter used is one or more of concentration, concentration time course, peak concentration, time after administration to peak concentration, terminal half-life, AUC, bioavailability, absorption, volume of distribution, metabolism, excretion, biotransformation, clearance or a combination thereof.

18. The method of claim 13, wherein the COX-2 inhibitor and the anti-hypertensive are administered in a fixed-dose combination.

19. The method of claim 13, wherein the COX-2 inhibitor is celecoxib.

20. The method of claim 13, wherein the anti-hypertensive is olmesartan.

21. The method of claim 13, wherein the predetermined PK parameters and the ranges for these PK parameters are identified after data reduction, but without clustering the data, and the differences between the ranges are statistically significant.

22. The method of claim 13, wherein the predetermined PK parameters and the ranges for these PK parameters are identified using data reduction and clustering, and the differences between the ranges are statistically significant.

23. The method of claim 13, wherein the predetermined PK parameters and the ranges for these PK parameters are identified using clustering without data reduction and the differences between the ranges are statistically significant.
